(19) United States
(12) Patent Application Publication
(10) Pub. No.: US 2023/0248411 A1
(43) Pub. Date: Aug. 10, 2023

(54) Cryotherapy System of Treatment for Ear, Nose, and Throat Disorders

(71) Applicant: Arrinex, Inc., Redwood City, CA (US)

(72) Inventors: William Jason Fox, San Mateo, CA (US); Brian Fahey, Menlo Park, CA (US); Vahid Saadat, Atherton, CA (US); William L. Gould, Santa Fe, NM (US); David Moosavi, Redwood City, CA (US)

(21) Appl. No.: 18/012,501

(22) PCT Filed: Sep. 28, 2021
(86) PCT No.: PCT/US2021/052355

§ 371 Date: Dec. 22, 2022

Related U.S. Application Data
(60) Provisional application No. 63/084,418, filed on Sep. 28, 2020.

(51) Int. Cl. A61B 18/02; A61B 90/00

(52) U.S. Cl. CPC A61B 18/0218; A61B 90/37; A61B 2018/00327

(57) ABSTRACT

In an example, a cryotherapy system includes a base station, a cryotherapy applicator, and a cryogen conduit configured to couple the cryotherapy applicator to the base station and supply a cryogen from the base station to the cryotherapy applicator. The base station includes a housing including a canister receptacle that is configured to receive a canister containing the cryogen. The cryotherapy applicator includes a handle, a shaft extending from the distal end of the handle, and an end-effector coupled to the shaft. The end-effector is configured to use the cryogen to ablate a target tissue. An entirety of the cryotherapy applicator is movable relative to an entirety of the base station while the cryogen conduit couples the cryotherapy applicator to the base station.

### CROSS-REFERENCE TO RELATED APPLICATION

The present application claims the benefit of U.S. Provisional Application No. 63/084,418 filed on Sep. 28, 2020, the contents of which is hereby incorporated by reference in its entirety.

### FIELD

The present disclosure generally relates to cryotherapy and, in particular, to cryotherapy systems and methods of treatment for ear, nose, and/or throat disorders.

### BACKGROUND

In general, thermal therapies involve treating tissue by inducing a temperature change that selectively induces alterations of the tissue, either temporarily or permanently. Depending on the tissue targeted for treatment, this thermal alteration may provide various benefits, including destroying the tissue and/or altering nerve signaling pathways. Ablation may be accomplished by applying heat (for example, with radiofrequency, laser, microwave, high intensity focused ultrasound (HIFU), or resistive heating methods) or by applying cooling energy (for example, using cryoablation techniques).

The term “cryotherapy” describes a class of thermal therapies that involve inducing a relatively cold temperature in a tissue, and includes therapies generally referred to as therapeutic hypothermia and cryoablation. Depending on the temperatures and exposure times involved, the clinical goals of various cryotherapies may range from improved tissue healing/recovery (e.g., as with therapeutic hypothermia employed during physical therapy sessions) to selective tissue damage or destruction (e.g., during cryoablation used for neuromodulation or tumor-destruction purposes). Any tissue alteration introduced during cryotherapy may be temporary or permanent, depending on the tissue treated and one or more characteristics of the therapy applied to the tissue.

Rhinitis is defined as inflammation of the membranes lining the nose, and is characterized by nasal symptoms including itching, rhinorrhea, and/or nasal congestion. Chronic rhinitis affects millions of people and is a leading cause for patients to seek medical care. Medical treatment has been shown to have limited effects for chronic rhinitis sufferers and requires daily medication use or onerous allergy treatments, and up to 20% of patients may be refractory. Selectively interrupting the Posterior Nasal Nerves (PNN), Accessory Posterior Nasal Nerves (APNN), and/or other nervous structures in patients with chronic rhinitis (e.g., by applying cryotherapy within the nasal cavity to cryoablate these nerves) has been shown to improve symptoms with limited to elimination of side effects.

Other disorders in the ear, nose, or throat can also be treated using cryotherapy.

### SUMMARY

In an example, a cryotherapy system includes a base station, a cryotherapy applicator, and a cryogen conduit. The base station includes a housing including a canister receptacle that is configured to receive a canister containing a cryogen. The housing defines an internal chamber. The base station also includes a cryogen outlet on an exterior surface of the housing. The cryogen outlet is configured to output the cryogen from the base station. The base station further includes a cryogen flow assembly in the internal chamber of the housing. The cryogen flow assembly is configured to supply the cryogen from the canister to the cryogen outlet. The base station also includes a controller configured to control a flow of the cryogen through the cryogen flow assembly from the canister to the cryogen outlet.

The cryotherapy applicator includes a handle that is configured to be gripped by a user during a cryotherapy procedure. The handle has a proximal end and a distal end. The cryotherapy applicator also includes a shaft extending from the distal end of the handle, and an end-effector coupled to the shaft. The end-effector is configured to use the cryogen to ablate a target tissue.

The cryogen conduit is configured to couple the cryotherapy applicator to the base station and supply the cryogen from the base station to the cryotherapy applicator. The cryogen conduit has (i) a first end extending from the proximal end of the handle of the cryotherapy applicator and (ii) a second end configured to couple to the cryogen outlet of the base station. An entirety of the cryotherapy applicator is movable relative to an entirety of the base station while the cryogen conduit couples the cryotherapy applicator to the base station.

In another example, a method of operating a cryotherapy system is described. The method includes coupling a canister containing a cryogen to a canister receptacle of a base station, and coupling, using a cryogen conduit, a cryotherapy applicator to a cryogen outlet on an exterior surface of a housing of the base station. The cryogen conduit has (i) a first end extending from a proximal end of a handle of the cryotherapy applicator and (ii) a second end configured to couple to the cryogen outlet of the base station.

The cryotherapy applicator includes the handle that is configured to be gripped by a user during a cryotherapy procedure. The handle has the proximal end and a distal end. The cryotherapy applicator also includes a shaft extending from the distal end of the handle, and an end-effector coupled to the shaft. The end-effector is configured to use the cryogen to ablate a target tissue.

The method also includes, while the cryogen conduit couples the cryotherapy applicator to the base station, moving an entirety of the cryotherapy applicator relative to an entirety of the base station to insert the end-effector in a nasal cavity and navigate the end-effector to the target tissue. After navigating the end-effector to the target tissue, the method includes supplying the cryogen from the canister in the base station to the end-effector to ablate the target tissue.

The features, functions, and advantages that have been discussed can be achieved independently in various embodiments or may be combined in yet other embodiments further details of which can be seen with reference to the following description and drawings.

### BRIEF DESCRIPTION OF THE FIGURES

The novel features believed characteristic of the illustrative embodiments are set forth in the appended claims. The illustrative embodiments, however, as well as a preferred mode of use, further objectives and descriptions thereof, will best be understood by reference to the following detailed description of an illustrative embodiment of the present disclosure when read in conjunction with the accompanying drawings, wherein:

FIG. 1 depicts a simplified block diagram of a cryotherapy system, according to an example.

FIG. 2 depicts a simplified block diagram of a cryotherapy system, according to an example.

FIG. 3A depicts a first canister coupled to a canister receptacle, according to an example.

FIG. 3B depicts a second canister coupled to the canister receptacle of FIG. 3A, according to an depicts.

FIG. 3C illustrates a second canister coupled to the canister receptacle of FIG. 3A, according to an example.

FIG. 4 depicts a canister receptacle coupled to a plurality of canisters, according to another example.

FIG. 5 depicts a partial perspective view of an implementation of the cryotherapy system shown in FIG. 2, according to an example.

FIG. 6 depicts a perspective view of a base station for the cryotherapy system shown in FIG. 5, according to an example.

FIG. 7 depicts another perspective view of the base station shown in FIG. 6, according to an example.

FIG. 8 depicts a perspective view of an implementation of a cryotherapy applicator and a camera for the cryotherapy system shown in FIG. 5, according to an example.

FIG. 9 depicts a perspective view of an implementation of the cryotherapy applicator shown in FIG. 8, according to an example.

FIG. 10 depicts a flowchart for a method of operating a cryotherapy system, according to an example.

FIG. 11 depicts a flowchart of a method of operating a cryotherapy system that can be used with the method of FIG. 10, according to an example.

FIG. 12 depicts a flowchart of a method of operating a cryotherapy system that can be used with the method of FIG. 10, according to an example.

FIG. 13 depicts a flowchart of a method of operating a cryotherapy system that can be used with the method of FIG. 10, according to an example.

FIG. 14 depicts a flowchart of a method of operating a cryotherapy system that can be used with the method of FIG. 10, according to an example.

FIG. 15 depicts a flowchart of a method of operating a cryotherapy system that can be used with the method of FIG. 10, according to an example.

### DETAILED DESCRIPTION

Disclosed embodiments will now be described more fully hereinafter with reference to the accompanying drawings, in which some, but not all of the disclosed embodiments are shown. Indeed, several different embodiments may be described and should not be construed as limited to the embodiments set forth herein. Rather, these embodiments are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

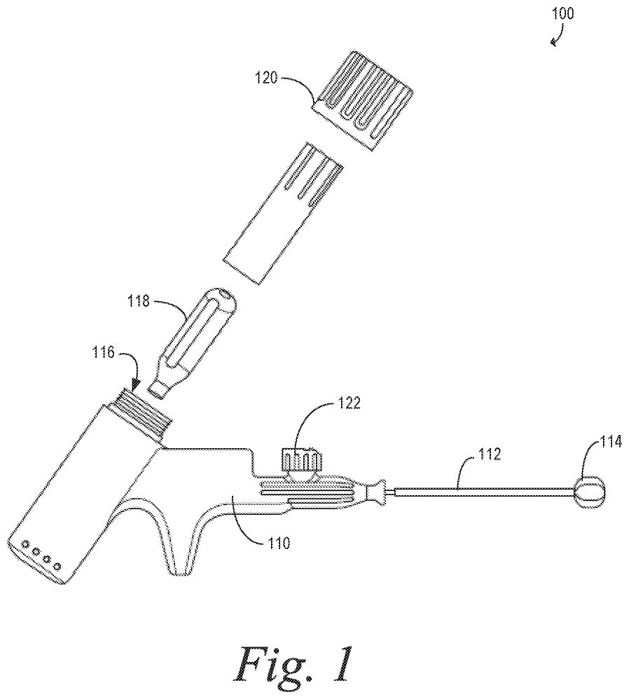

FIG. 1 depicts an existing cryotherapy system 100 that can be used to treat an ear, a nose, and/or a throat with cryotherapy. As shown in FIG. 1, the cryotherapy system 100 includes a housing 110, an elongated shaft 112, and an end-effector 114. The housing 110 includes a canister receptacle 116 that can receive a canister 118, which stores a cryogen. The housing 110 also includes a user input device 122 that can control a flow of the cryogen from the canister 118 in the canister receptacle 116 to the end-effector 114. In general, each canister 118 may include a quantity of the cryogen that is sufficient to perform cryotherapy on a single target tissue.

In this arrangement, when the cryotherapy treatment is to be performed on multiple target tissues, a medical practitioner has to remove a first canister 118 and insert a second canister 118. For instance, a procedure for treating target tissues in two nostrils of a patient may include the following steps according to one example. First, the medical practitioner may remove a cap 120 from the housing 110, insert a first canister 118 into the canister receptacle 116, and then recouple the cap 120 to the housing 110. The medical practitioner can then grasp the housing 110 and insert the end-effector 114 and the elongated shaft 112 into a first nostril of the patient, and navigate the end-effector 114 to a first target tissue in the first nostril. For some procedures, the medical practitioner may perform the insertion and navigation while holding the housing 110 with one hand, and holding a separate endoscope with the other hand. The endoscope generally has a rigid shaft that extends along and adjacent to the elongated shaft 112 of the cryotherapy system 100.

While the end-effector 114 is located at the first target tissue, the user input device 122 is actuated to supply the cryogen from the first canister 118 to the end-effector 114, which uses the cryogen to apply the cryotherapy to the first target tissue. In procedures in which the medical practitioner holds the housing 110 in one hand and the endoscope in the other hand, a second medical practitioner may assist by actuating the user input device 122. After applying cryotherapy to the first target tissue, the end-effector 114 (and the endoscope) may be removed from the first nostril.

Also, after performing the cryotherapy on the first target tissue, the first canister 118 is be depleted of sufficient cryogen to treat the second target tissue. As such, the medical practitioner may remove the first canister 118 from the canister receptacle 116, and insert a second canister 118 into the canister receptacle 116. The medical practitioner can then grasp the housing 110 and insert the end-effector 114 and the elongated shaft 112 into a second nostril of the patient, and navigate the end-effector 114 to a second target tissue in the second nostril. After the end-effector 114 is located at the second target tissue, the user input device 122 is actuated again to supply the cryogen from the second canister 118 to the end-effector 114, which uses the cryogen to apply cryotherapy to the second target tissue in the second nostril. In some implementations, the entire cryotherapy system 100 may be disposed of after treating the first target tissue in the first nostril and the second target tissue in the second nostril.

Although the cryotherapy system 100 shown in FIG. 1 and the example procedure for performing cryotherapy described above can effectively treat a variety of disorders and/or conditions in the ear, the nose, and/or the throat, the present disclosure provides cryotherapy systems and methods that can help to improve aspects of treating tissue with cryotherapy.

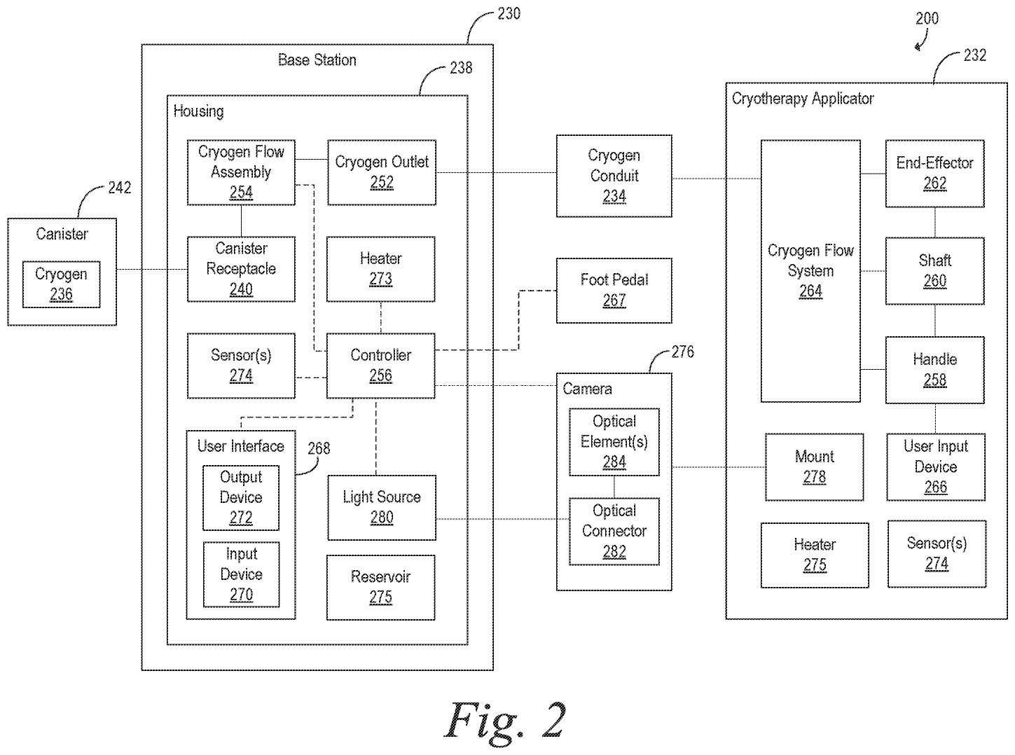

Referring now to FIG. 2, a cryotherapy system 200 is shown according to an example. As described in detail below, the cryotherapy system 200 includes a plurality of components that provide for treating a target tissue with cryotherapy to alter the target tissue by, for example, destroying the target tissue and/or altering nerve signaling pathways. In one example, the cryotherapy system 200 can apply cryotherapy to one or more target tissues such as, for instance, Posterior Nasal Nerves (PNN), Accessory Posterior Nasal Nerves (APNN), sphenopalatine ganglion, pterygopalatine ganglion, and/or other nervous structures to treat rhinitis. In other examples, the cryotherapy system 200 can apply cryotherapy to nerves in the nose to treat chronic headaches. In still other examples, the cryotherapy system 200 can apply cryotherapy to a sphenopalatine region. In other examples, the cryotherapy system 200 can apply cryotherapy to other target tissues in the ear, the nose, and/or the throat.

As shown in FIG. 2, the cryotherapy system 200 includes a base station 230, a cryotherapy applicator 232, and a cryogen conduit 234 that can couple the cryotherapy applicator 232 to the base station 230. The base station 230 can store a cryogen 236 and supply the cryogen 236 to the cryotherapy applicator 232 via the cryogen conduit 234. The cryotherapy applicator 232 can receive the cryogen 236 from the cryogen conduit 234 and use the cryogen 236 to apply therapeutic hypothermia and/or cryoablate the target tissue in an ear, a nose, and/or a throat of a patient.

Within examples, an entirety of the cryotherapy applicator 232 is movable relative to an entirety of the base station 230 while the cryogen conduit 234 couples the cryotherapy applicator 232 to the base station 230. For instance, the cryogen conduit 234 can have a flexibility that allows the cryotherapy applicator 232 to move relative to the base station 230. In an implementation, the cryotherapy applicator 232 can move with six degrees of freedom relative to the base station 230 (e.g., including three degrees of freedom for translation and three degrees of freedom for rotation). This can facilitate navigating the cryotherapy applicator 232 around bone and tissue structures in the ear, the nose, and/or the throat to reach the target tissue. However, in other examples, the cryotherapy applicator 232 can move with less than six degrees of freedom relative to the base station 230.

In this arrangement, a medical practitioner can hold and move the cryotherapy applicator 232 relative to the patient while the base station 230 remains stationary relative to the patient. This can help to solve a number of technical challenges that may be encountered with existing cryotherapy systems.

For example, existing cryotherapy systems generally require the medical practitioner to hold and support all of the components of the cryotherapy systems in their hand while performing the cryotherapy procedure. By contrast, the components of the cryotherapy system 200 shown in FIG. 1 are distributed between (i) the base station 230, which is not held by the medical practitioner during the cryotherapy procedure, and (ii) the cryotherapy applicator 232, which is held and manipulated by the medical practitioner during the cryotherapy procedure. As such, the cryotherapy applicator 232 can be implemented with reduced weight and/or improved ergonomics as compared to existing cryotherapy systems (e.g., the cryotherapy system 100 shown in FIG. 1).

Additionally, as described below, providing a first set of the components in the base station 230 and a second set of components in the cryotherapy applicator 232 can allow the cryotherapy system 200 to include features that may be too large to be practical in existing cryotherapy systems in which the medical practitioner holds all components in their hand(s) at the same time while performing the cryotherapy procedure (e.g., the cryotherapy system 100 shown in FIG. 1).

As shown in FIG. 1, the base station 230 includes a housing 238 that defines an internal chamber in which one or more components of the cryotherapy system 200 can be housed. The housing 238 can include a canister receptacle 240 that can receive a canister 242 containing a cryogen 236. As examples, the cryogen 236 can include a nitrous oxide and/or nitrogen. Also, as examples, the canister 242 can include a container that can store the cryogen 236 in a compressed manner (e.g., at a pressure that is greater than an atmospheric pressure of an environment that is external to the canister 242).

Because the base station 230 includes the canister receptacle 240 and the base station 230 is not held by the medical practitioner while performing the cryotherapy treatment on the patient, the canister 242 can store a greater amount of the cryogen 236 than existing cryotherapy systems in which the medical practitioner holds the cryogen canister while performing the cryotherapy treatment. In an example, the canister 242 can contain an amount of the cryogen 236 that is greater than or equal to an amount of the cryogen 236 that is used to treat at least two target tissues. This can allow the medical practitioner to more efficiently treat a plurality of target tissues as the medical practitioner does not need to swap out the canister 242 after treating each target tissue.

In one example implementation, the canister 242 can contain an amount of the cryogen 236 that is greater than or equal to an amount of cryogen 236 that is used to treat a first target tissue in a first nostril and a second target tissue in a second nostril. For instance, in one existing cryotherapy system (e.g., the cryotherapy system 100 shown in FIG. 1), the canister includes 10 milliliters (mL) of the cryogen, which can be a sufficient amount of the cryogen 236 to treat one target tissue in one nostril of a patient. With the canister receptacle 240 in the base station 230, the canister 242 of the cryotherapy system 200 can include a greater range of sizes and store a greater range of amounts of the cryogen 236.

In one example, the canister 242 can contain greater than 10 mL of the cryogen 236. As noted above, this can allow the canister 242 to store an amount of the cryogen 236 that can be used to treat a first target tissue in a first nostril of a patient and a second target tissue in a second nostril of the patient without changing the canister 242 between these treatments. In another example, the canister 242 can contain between approximately 10 mL and approximately 32 mL of the cryogen 236 (e.g., approximately 20 mL). In another example, the canister 242 can contain between approximately 32 ml and approximately 60 liters (L) of the cryogen 236. This can allow the cryotherapy system 200 to store an amount of the cryogen 236 that can be used to treat a relatively large number of target tissues (e.g., including a plurality of target tissues on one patient and/or a plurality of target tissues over a plurality of patients). Although it may be beneficial to provide the canister 242 with relatively greater volumes to store relatively greater amounts of the cryogen 236, the canister 242 can store a quantity of the cryogen 236 that is approximately less than or equal to 10 mL in some instances.

In one example, the canister receptacle 240 can be configured to receive only a single type of the canister 242 having a single size. In other examples, the canister receptacle 240 can be configured to receive (i) a first canister 242 having a first size and (ii) a second canister 242, which has a second size that is greater than the first size of the first canister 242. As such, the first canister 242 can contain a first volume of the cryogen 236 and the second canister 242 can contain a second volume of the cryogen 236, where the second volume is greater than the first volume.

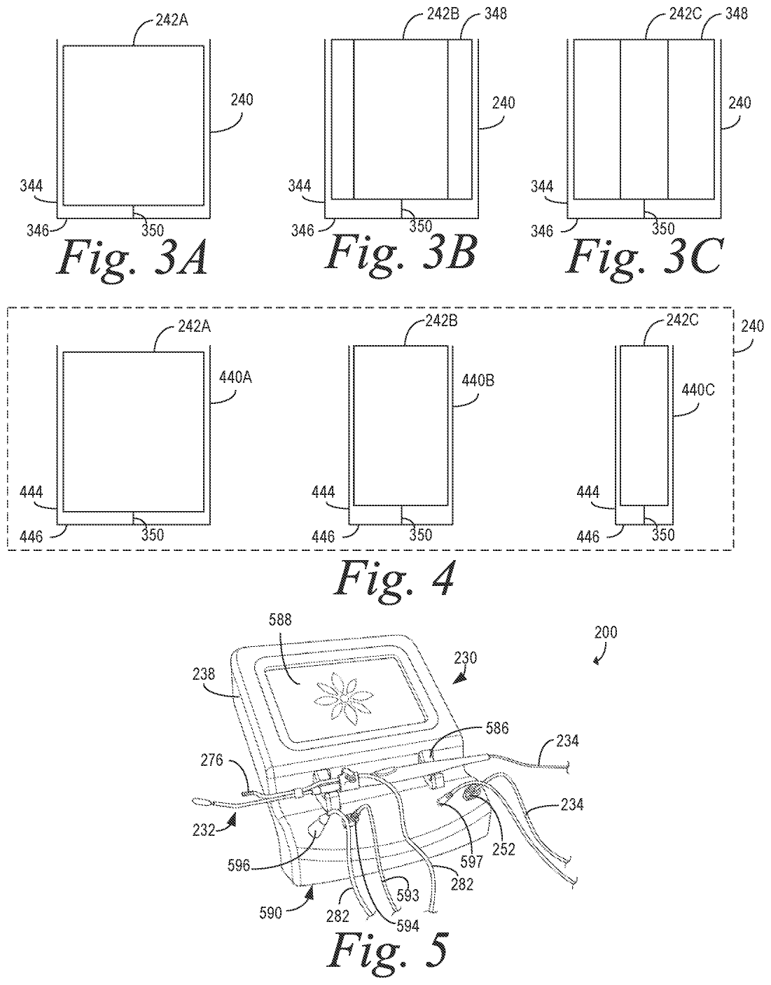

FIGS. 3A-3C and FIG. 4 depict implementations of the canister receptacle 240 that can receive a plurality of differently sized canisters 242A-242C, according to some examples. In FIGS. 3A-3C, the canister receptacle 240 has a lateral wall 344 and an end wall 346 that define an internal cavity in which the canisters 242A-242C can be received. FIG. 3A depicts the canister receptacle 240 receiving a first canister 242A having a first size and storing a first volume of the cryogen 236, FIG. 3B depicts the canister receptacle 240 receiving a second canister 242B having a second size and storing a second volume of the cryogen 236, and FIG. 3C depicts the canister receptacle 240 receiving a third canister 242C having a third size and storing a third volume of the cryogen 236.

As shown in FIGS. 3A-3C, a single canister receptacle 240 of the base station 230 can receive the first canister 242A, the second canister 242B, and the third canister 242C despite the different sizes of the first canister 242A, the second canister 242B, and the third canister 242C. Additionally, as shown in FIGS. 3A-3C, the cryotherapy system 200 can include one or more adapters 348 to facilitate coupling at least some of the canisters 242A-242C with the canister receptacle 240. The adapter(s) 348 can help improve an amount of support provided by the lateral wall 344 to the canisters 242B-242C.

For instance, in FIGS. 3B-3C, the adapter 348 includes a bore in which the canister 242 is received. A circumference of the bore of the adapter 348 can generally correspond to a circumference of the canister 242B, 242C to help support the canister 242B, 242C in the adapter 348. As such, the bore of the adapter 348 in FIG. 3B can have a circumference that is greater than a circumference of the bore of the adapter 348 in FIG. 3C to accommodate the relatively larger circumference of second canister 242B relative to the third canister 242C. Additionally, as shown in FIGS. 3B-3C, the adapter 348 can have an outer circumference that generally corresponds to a circumference of the lateral wall 344 of the canister receptacle 240. This can also help to support the adapter 348 and the canister 242B, 242C in the internal cavity of the canister receptacle 240.

FIG. 4 depicts another implementation in which the canister receptacle 240 includes a plurality of cartridge receptacles 440A-440C, which each define a respectively internal cavity for receiving a respective type of canister 242A-242C. For example, as shown in FIG. 4, the plurality of cartridge receptacles 440A-440C can include a first canister receptacle 440A, a second canister receptacle 440B, and a third canister receptacle 440C. The first canister receptacle 440A can have a first lateral wall 444 that defines a first internal cavity having a first size and/or a first shape that corresponds to a size and/or a shape of the first canister 242A, the second canister receptacle 440B can have a second lateral wall 444 that defines a second internal cavity having a second size and/or a second shape that corresponds to a size and/or a shape of the second canister 242B, and the third canister receptacle 440C can have a first lateral wall 444 that defines a third internal cavity having a third size and/or a third shape that corresponds to a size and/or a shape of the third canister 242C. In this way, the first internal cavity of the first canister receptacle 440A can be configured to receive the first canister 242A, the second internal cavity of the second canister receptacle 440B can be configured to receive the second canister 242B, and the third internal cavity of the third canister receptacle 440C can be configured to receive the third canister 242C.

Although FIGS. 3A-3C and FIG. 4 depict examples in which the canister receptacle 240 can couple to three differently configured canisters 242A-242C, the canister receptacle 240 can be configured to couple to a lesser or a greater quantity of types of canisters 242A-242C in other examples (e.g., one type of canister 242, two types of canisters 242, four types of canisters 242, etc. where each type of canister 242 has a respective configuration that is different from the other types of canisters 242 with respect to at least one of a size of the canister 242 or a shape of the canister 242).

Additionally, in FIGS. 3A-3C and FIG. 4, the canister receptacle 240 includes a pin 350 at the end wall 346, 446 that is configured to puncture a wall of the canister 242A-242C to fluidly couple the canister 242A-242C with the canister receptacle 240 and provide for egress of the cryogen 236 to the base station 230. In another example, the canister receptacle 240 and the canister receptacle 240 can additionally or alternatively be coupled by at least one of coupling arrangement selected from a group consisting of: a friction fit coupling and a threaded engagement coupling.

As described, the canister 242 and the canister receptacle 240 of the base station 230 can be configured such that the canister 242 is removably coupled to the base station 230 to provide a quantity of the cryogen 236 for use during one or more cryotherapy treatments. In an alternative example, the canister 242 can be permanently coupled to the canister receptacle 240. In such alternative examples, the canister 242 can be a refillable structure that is at a fixed position in the housing 238 of the base station 230.

Referring again to FIG. 2, the base station 230 also includes a cryogen outlet 252 on an exterior surface of the housing 238, and a cryogen flow assembly 254 in the internal chamber of the housing 238. The cryogen flow assembly 254 is configured to supply the cryogen 236 from the canister 242 in the canister receptacle 240 to the cryogen outlet 252, and the cryogen outlet 252 is configured to output the cryogen 236 from the base station 230.

As examples, the cryogen flow assembly 254 can include one or more valves and/or one or more lumens that define a flow path for the cryogen 236 between the canister receptacle 240 and the cryogen outlet 252. The one or more valves can be operable to control a flow of the cryogen 236 through the lumen(s). In one example, each valve can be actuatable between (i) a closed state in which the valve prevents the cryogen 236 from flowing through the lumen(s) from the canister receptacle 240 to the cryogen outlet 252, and (ii) an open state in which the valve allows the cryogen 236 to flow through the lumen(s) from the canister receptacle 240 to the cryogen outlet 252. In this arrangement, the valve of the cryogen flow assembly 254 can be in the closed state prior to and/or after applying cryotherapy to the target tissue, and the valve of the cryogen flow assembly 254 can be in the open state while applying the cryotherapy to the target tissue during a cryotherapy treatment.

In some examples, the cryogen flow assembly 254 can have only the closed state and the open state. This can help to simplify the design in implementations in which the cryogen 236 may be substantially supplied at the cryogen outlet 252 at a constant flow rate. However, in other examples, the cryogen flow assembly 254 can be configured to supply the cryogen 236 at a plurality of flow rates. In such examples, for instance, the valve(s) of the cryogen flow assembly 254 can have a plurality of intermediate states between the closed state and the open state in which each intermediate state provides a respective flow rate of the plurality of flow rates. Supplying the cryogen 236 at a flow rate selected from among a plurality of flow rates can help to more precisely and granularly control the thermal energy applied to the target tissue during the cryotherapy treatment.

In some examples, the base station 230 can include a controller 256 that is configured to control a flow of the cryogen 236 through the cryogen flow assembly 254 from the canister 242 to the cryogen outlet 252. For example, the controller 256 can be in communication with the one or more valves of the cryogen flow assembly 254 an the controller 256 can be operable to transmit a control signal to the valve(s) to cause valve(s) to actuate to a selected state from among a plurality of states (e.g., the open state, the intermediate state(s), and the closed state) to control the flow of the cryogen 236 through the cryogen flow assembly 254. As described in further detail below, the controller 256 can be configured to transmit the control signal responsive to at least one trigger selected from among a group of triggers consisting of: a user input and/or a sensor signal from a sensor of the cryotherapy system 200.

The controller 256 can be implemented using hardware, software, and/or firmware. For example, the controller 256 can include one or more processors and a non-transitory computer readable medium (e.g., volatile and/or non-volatile memory) that stores machine language instructions or other executable instructions. The instructions, when executed by the one or more processors, may cause the controller 256 to carry out the various operations of the cryotherapy system 200 described herein.

As noted above, the cryogen conduit 234 is configured to couple the cryotherapy applicator 232 to the base station 230 and supply the cryogen 236 from the base station 230 to the cryotherapy applicator 232. For example, the cryogen conduit 234 can have (i) a first end extending from a proximal end of a handle 258 of the cryotherapy applicator 232 and (ii) a second end configured to couple to the cryogen outlet 252 of the base station 230. In one example, the first end of the cryogen conduit 234 can be fixedly coupled to the handle 258 of the cryotherapy applicator 232 whereas the second end of the cryogen conduit 234 can be removably coupled to the cryogen outlet 252 of the base station 230. In another example, the first end of the cryogen conduit 234 can be removably coupled to the handle 258 of the cryotherapy applicator 232 whereas the second end of the cryogen conduit 234 can be fixedly coupled to the cryogen outlet 252 of the base station 230. In yet another example, the first end of the cryogen conduit 234 can be removably coupled to the handle 258 of the cryotherapy applicator 232 whereas the second end of the cryogen conduit 234 can be removably coupled to the cryogen outlet 252 of the base station 230.

Removably coupling at least one of the first end or the second end of the cryogen conduit 234 to the handle 258 of the cryotherapy applicator 232 or the cryogen outlet 252 of the base station 230, respectively, can allow for the base station 230 to be used with a plurality of cryotherapy applicators 232. This can facilitate reusing the base station 230 for a plurality of treatments and/or a plurality of patients with different cryotherapy applicators 232. Additionally, this can provide for the cryotherapy applicator 232 to be fabricated in a manner that allows for the cryotherapy applicator to be disposable while the base station 230 is reusable. Additionally or alternatively, the removable coupling(s) can facilitate using the base station 230 with a plurality of cryotherapy applicators 232 having different configurations (e.g., a different size, a different shape, and/or a different material properties relative to each other), as described in further detail below.

As examples, the first end and/or the second end of the cryogen conduit 234 can be permanently coupled to the handle 258 of the cryotherapy applicator 232 and/or the cryogen outlet 252 of the base station 230 by welding, an adhesive, a barb fitting, and/or another form of coupling that cannot be repeatedly decoupled and recoupled by a medical practitioner. As another example, the permanent coupling can be provided by integrally forming at least a portion of the cryogen conduit 234 with at least a portion of base station 230 and/or the cryotherapy applicator 232. Also, as examples, the first end and/or the second end of the cryogen conduit 234 can be removably coupled to the handle 258 of the cryotherapy applicator 232 and/or the cryogen outlet 252 of the base station 230 by a threaded engagement coupling, a bayonet connector coupling, a quick-connect coupling, and/or a friction fit coupling.

As shown in FIG. 2, the cryotherapy applicator 232 can include the handle 258, a shaft 260, and an end-effector 262. The cryotherapy applicator 232 can also include a cryogen flow system 264, which includes at least one lumen that extends through the handle 258 and the shaft 260 from the first end of the cryogen conduit 234 to the end-effector 262. In this arrangement, the lumen(s) of the cryogen flow system 264 can supply the cryogen 236 received from the cryogen conduit 234 to the end-effector 262, and the end-effector 262 can use the cryogen 236 to apply thermal energy to the target tissue for the cryotherapy treatment.

In general, the handle 258 can be configured to facilitate a user gripping and manipulating the cryotherapy applicator 232 while performing cryotherapy. For example, the handle 258 can have a shape and/or a size that can facilitate a user performing cryotherapy by manipulating the cryotherapy applicator 232 using a single hand. In one implementation, the handle 258 can have a shape and/or a size that facilitates the user holding the cryotherapy applicator 232 in a writing utensil gripping manner (e.g., the handle 258 can have an axis that is substantially parallel to an axis of the shaft 260). For instance, the handle 258 of the cryotherapy applicator 232 can be elongated along a longitudinal axis such that the handle 258 is configured to be held by the user using a pencil grip. In another implementation, the handle 258 can have a shape and/or a size that facilitates the user holding the handle 258 in a pistol gripping manner (e.g., the handle 258 can have an axis that is transverse to an axis of the shaft 260). Additionally or alternatively, the handle 258 can facilitate gripping and manipulating the cryotherapy applicator 232 by having a shape and/or a size that is greater than a shape and/or a size of the shaft 260.

The shaft 260 can be configured to be at least partially inserted in a body cavity of a patient, where the body cavity includes a cavity in an ear, a nose, or a throat of the patient. For instance, the shaft 260 can be elongated along a longitudinal axis between a proximal end and a distal end of the shaft. In this arrangement, the proximal end of the shaft 260 can extend from a distal portion of the housing, and the distal end of the shaft 260 can be coupled to the end-effector 262.

In one example, the shaft 260 can have a diameter between approximately 1 mm and approximately 4 mm. Additionally, for example, the shaft 260 can be made from stainless Steel and/or semi-rigid polymer (e.g., such as Nylon or Pebax).

As noted above, the end-effector 262 is configured to use the cryogen 236 to apply thermal energy to the target tissue. In one example, the end-effector 262 can include a balloon into which the cryogen 236 (e.g., in the form of a compressed liquid) can expand as a gas. As another example, the end-effector 262 can include a metallic plate, which can be chilled through contact with the cryogen 236 (e.g., in the form of a circulating cooled fluid). In these examples, the end-effector 262 includes an intermediary feature (e.g., the balloon and/or the metallic plate) that transfers the thermal energy from the cryogen 236 to the target tissue. This can beneficially help to improve the uniformity of the distribution of cold temperatures applied across a targeted region of tissue. This indirect application of cooling can also prevent the cryogen 236 from direct exposure to the body in unwanted regions.

In some implementations, the end-effector 262 can have an active surface that is configured for contacting the target tissue and an inactive surface that is configured to positioned at or adjacent to another tissue. For example, the end-effector 262 include the active surface and an inactive surface such that the end-effector 262 applies the thermal energy to the target tissue contacting the active surface and does not apply the thermal energy to other tissue contacting the inactive surface. This can help to apply thermal energy in a relatively targeted manner to treat a specific target tissue.

In other implementations, an entirety of the end-effector 262 can be active such that the end-effector 262 applies the thermal energy omni-directionally. This can help to apply the thermal energy more broadly and, in some instances, can help to reduce a time for performing a cryotherapy procedure.

As shown in FIG. 2, the cryotherapy applicator 232 can also include a user input device 266 on the handle 258. The user input device 266 can control a flow of the cryogen 236 from the base station 230 to the end-effector 262. For instance, the user input device 266 can include one or more knobs, one or more triggers, one-or more buttons, one or more switches, one or more levers, and/or one or more dials that can be actuated to start a flow of the cryogen 236, stop a flow of the cryogen 236, increase a flow rate of the cryogen 236, and/or decrease the flow rate of the cryogen 236 from the base station 230 to the end-effector 262.

In one example, the user input device 266 is configured to transmit to a control signal to the controller 256 to cause the controller 256 to control the flow of the cryogen 236 to the end-effector 262. In another example, the user input device 266 can be configured to mechanically actuate a valve of the cryogen flow system 264 and/or a valve of the cryogen flow assembly 254 to control the flow of the cryogen 236 from the base station 230 to the end-effector 262.

In some examples, the cryotherapy system 200 can include a foot pedal 267 that can be operable to control the flow of the cryogen 236 in addition or in alternative to the user input device 266 of the cryotherapy applicator 232. As shown in FIG. 2, the foot pedal 267 can be in communication with the controller 256 of the base station 230. In this arrangement, the foot pedal 267 can be configured to transmit a control signal to the controller 256 to cause the controller 256 to control the flow of cryogen 236. For example, the foot pedal 267 can be actuated to start a flow of the cryogen 236, stop a flow of the cryogen 236, increase a flow rate of the cryogen 236, and/or decrease the flow rate of the cryogen 236 from the base station 230 to the end-effector 262. In implementations that include the foot pedal 267, the foot pedal 267 can help to make it easier to hold the cryotherapy applicator 232 at the target tissue while supplying the cryogen 236 to the end-effector 262.

In some examples, the base station 230 can include a user interface 268 that can be operable to control the flow of the cryogen 236 in addition or in alternative to the user input device 266 of the cryotherapy applicator 232 and/or the foot pedal 267. For instance, as shown in FIG. 2, the user interface 268 can include a base-station input device 270 that is configured to receive one or more inputs from a user. As examples, the base-station input device 270 can include one or more switches, one or more push buttons, one or more levers, one or more touchscreen displays, and/or one or more microphones on the housing 238 of the base station 230 for receiving the user input. The base-station input device 270 can be in communication with the controller 256 and configured to provide the user input to the controller 256.

In this arrangement, the controller 256 can receive the user input from the base-station input device 270 and the controller 256 can perform one or more actions responsive to the one or more inputs received via the base-station input device 270. As examples, the one or more actions can include at least one action selected from a group of actions consisting of: (i) starting a flow of the cryogen 236, (ii) stopping the flow of the cryogen 236, (iii) increasing a flow rate of the cryogen 236, and (iv) decreasing a flow rate of the cryogen 236. In one implementation, the base-station input device 270 can cause the controller 256 to set a flow rate of the cryogen 236, and then the user input device 266 on the cryotherapy applicator 232 and/or the foot pedal 267 can be used to start the flow of the cryogen 236 at the flow rate that was set using the base-station input device 270.

In some examples, the base-station input device 270 can additionally or alternatively indicate to the controller 256 an amount of time for which the cryogen 236 will flow during a treatment. For instance, the controller 256 can implement a timer that the controller 256 can use to make a determination that the cryogen 236 has been supplied to the end-effector 262 for the amount of time indicated by the base-station input device 270 and, responsive to the determination, automatically stop supplying the cryogen 236 to the end-effector 262. This can help a medical practitioner to control an amount of thermal energy applied to the target tissue.

As shown in FIG. 2, the user interface 268 can additionally or alternatively include an output device 272 that is configured to output information to a user. For example, the output device 272 can include one or more speakers, one or more indicator lights, and/or one or more display devices that are configured to provide visual outputs and/or auditory outputs to the user. In one implementation, the output device 272 and the base-station input device 270 can be combined in the form of a touchscreen that can receive a user input from the user and output information to the user. As shown in FIG. 2, the output device 272 can be communicatively coupled to the controller 256 and the controller 256 can cause the output device 272 to output information to the user.

In some examples, the output device 272 can output data relating to personal information for a patient. For instance, the personal information can include at least one item of information selected from a group including: a name of the patient, a birthdate of the patient, an age of the patient, a gender of the patient, a height of the patient, a weight of the patient, a body temperature of the patient, a heart rate of the patient, a blood pressure of the patient, and an oxygen level of the patient. Outputting this information via the output device 272 can efficiently and conveniently provide the medical practitioner with information that can be used to set operational parameters (e.g., the rate of flow and/or a timer duration) and/or confirm operational parameters of the cryotherapy system 200 for a given cryotherapy treatment.

In some examples, the output device 272 can additionally or alternatively output data relating to procedure information. For instance, the procedure information can include at least one item of information selected from a group including: a type of procedure, an identification of a type of target tissue (e.g., a type of nerve), and a disorder to be treated. Outputting this information via the output device 272 can also efficiently and conveniently provide the medical practitioner with information that can be used to set operational parameters (e.g., the rate of flow and/or a timer duration) and/or confirm operational parameters of the cryotherapy system 200 for a given cryotherapy treatment.

In some examples, the output device 272 can additionally or alternatively output data relating to a status of the cryotherapy system 200. For instance, the output device 272 can output data relating to at least one item of information selected from a group including: (i) an indication that the cryogen 236 is flowing from the base station 230 to the end-effector 262, (ii) an indication that the cryogen is not flowing from the base station 230 to the end-effector 262, (iii) a flow rate of the cryogen 236 flowing from the base station 230 to the end-effector 262 (e.g., a value expressed in terms of units of volume per unit of time), (iv) a timer indicating a time that has elapsed since the cryogen 236 started flowing from the base station 230 to the end-effector 262, and (v) a timer indicating a time remaining until the flow of the cryogen 236 will be stopped. This information can provide feedback to the medical practitioner that can help to perform the cryotherapy treatment.

In some examples, the output device 272 can additionally or alternatively output data that is based on information determined by one or more sensors 274 of the cryotherapy system 200. As shown in FIG. 2, the one or more sensors 274 can be provided as a part of the base station 230 and/or a part of the cryotherapy applicator 232. As examples, the one or more sensors 274 can include at least one sensor selected from a group of sensors including: a temperature sensor (e.g., a thermistor, a thermocouple, and/or a resistance thermal detector), a pressure sensor (e.g., a strain gage, a piezoelectric sensor, and/or a force sensitive resistor), an ultrasonic sensor (e.g., an ultrasonic Doppler flow sensor and/or intranasal ultrasound imaging), an optical sensor (e.g., an optical Doppler flow sensor, and/or an infrared sensor configured to use infrared wavelengths to view vasculature), and an electrode sensor (e.g., one or more stimulation/response electrodes, and/or a polar electrode array that is configured to measure at least one of: complex impedance and conductivity). The one or more sensors 274 can be communicatively coupled to the controller 256.

In this arrangement, the one or more sensors 274 can be configured to sense a condition and transmit to the controller 256 a sensor signal indicative of the condition sensed by the one or more sensors 274. The controller 256 can receive the sensor signal and, based on the sensor signal, responsively perform one or more actions such as, for instance, displaying information based on the sensor signal (e.g., displaying a temperature, a flow rate, and/or a pressure sensed by the sensor(s) 274), and/or operating the cryogen flow assembly 254 and/or the cryogen flow system 264 of the cryotherapy system 200 based on the sensor signal.

In some examples, the one or more sensors 274 can include a temperature sensor that can sense a temperature that is indicative of a temperature of the cryogen 236 in the canister 242. In some instances, the temperature of the cryogen 236 can affect the flow rate of the cryogen 236. In some implementations, the output device 272 can output an indication of the temperature of the cryogen 236 based on the temperature sensed by the temperature sensor.

In some implementations, the controller 256 can additionally or alternatively take an action to adjust a temperature of the cryogen 236 in the canister 242. For instance, as shown in FIG. 2, the base station 230 can include a heater 273 that is configured to increase a temperature of the canister 242 in the canister receptacle 240. As an example, the heater 273 can include an electric resistive heating device that can transduce electrical energy into thermal energy for heating the canister 242. Additionally, as one example, the heater 273 can be arranged with the canister receptacle 240 such that the heater 273 can direct heat into an internal cavity of the canister receptacle 240 in which the canister 242 is received (e.g., the heater 273 can be at or adjacent to the lateral wall 344, 444 and/or the end wall 346, 446 of the example canister receptacle 240 shown in FIGS. 3A-4).

In this arrangement, the controller 256 can be configured to, based on the sensor signal, cause the heater 273 to increase the temperature of the canister 242 of the canister receptacle 240. In one example, the controller 256 can perform a comparison of the temperature sensed by the temperature sensor to a threshold temperature and, based on the comparison, determine that the temperature sensed by the temperature sensor is less than the threshold temperature. Responsive to determining that the temperature sensed by the temperature sensor is less than the threshold temperature, the controller 256 can cause the heater 273 to increase the temperature of the canister 242 until the controller 256 determines that the temperature sensed by the temperature sensor is at or above the threshold temperature.

In some examples, the one or more sensors 274 can include a temperature sensor located on an exterior of the shaft 260 at a location that is proximal to the end-effector 262. For instance, the temperature sensor located on the exterior of the shaft 260 and proximal to the end-effector 262 can help to determine if a cryogenic cooling treatment has expanded outside of a desired target area. For instance, if the temperature sensor senses a temperature below a threshold temperature, it may be indicative that the cryotherapy system 200 should cease supplying the cryogen 236 to the end-effector 262.

In one implementation, the controller 256 can perform a comparison of the temperature sensed by the temperature sensor to the threshold temperature and, based on the comparison, determine that the temperature sensed by the temperature sensor is less than the threshold temperature. Responsive to determining that the temperature sensed by the temperature sensor is less than the threshold temperature, the controller 256 can cause the output device 272 to provide an alarm in the form of an audio output and/or a visual output to indicate to the medical practitioner that the supply of the cryogen 236 should be stopped.

In some implementations, the controller 256 can be additionally or alternatively configured to automatically stop a supply and/or reduce a flow rate of the cryogen 236 to the end-effector 262 responsive to the temperature sensor sensing that the temperature is below the threshold temperature. For instance, responsive to the controller 256 determining that the temperature sensed by the temperature sensor is less than the threshold temperature, the controller 256 can automatically cause the cryogen flow assembly 254 of the base station 230 and/or the cryogen flow system 264 of the cryotherapy applicator 232 to stop supplying the cryogen 236 to the end-effector 262 and/or reduce a flow rate of the cryogen 236 to the end-effector 262.

In some examples, the one or more sensors 274 can include a temperature sensor located in an interior of the shaft 260 at a location that is proximal to the end-effector 262, and/or a temperature sensor located in an interior space of the end-effector 262 (e.g., in an interior space of a balloon of the end-effector 262). For instance, the temperature sensor(s) at these locations can sense a temperature that can be indicative of whether the cryogen 236 is being fully converted from a liquid phase to a gas phase.

In one implementation, the controller 256 can perform a comparison of the temperature sensed by the temperature sensor to the threshold temperature and, based on the comparison, determine that the temperature sensed by the temperature sensor is less than the threshold temperature (e.g., approximately 88 degrees Celsius). Responsive to determining that the temperature sensed by the temperature sensor is less than the threshold temperature, the controller 256 can cause the output device 272 to provide an alarm in the form of an audio output and/or a visual output to indicate to the medical practitioner that the cryogen 236 is not being fully converted from the liquid phase to the gas phase.

In some implementations, the controller 256 can be additionally or alternatively configured to automatically stop a supply and/or reduce a flow rate of the cryogen 236 to the end-effector 262 responsive to the temperature sensor sensing that the temperature is below the threshold temperature. For instance, responsive to the controller 256 determining that the temperature sensed by the temperature sensor is less than the threshold temperature, the controller 256 can automatically cause the cryogen flow assembly 254 of the base station 230 and/or the cryogen flow system 264 of the cryotherapy applicator 232 to stop supplying the cryogen 236 to the end-effector 262 and/or reduce a flow rate of the cryogen 236 to the end-effector 262.

In some examples, the one or more sensors 274 can include a temperature sensor located in an exterior surface of the end-effector 262 (e.g., on a treatment side of the end-effector 262 that is placed into contact with the target tissue during a cryotherapy procedure). In an example, the temperature sensor located on the exterior surface of the end-effector 262 can measure a temperature that can be indicative of an effectiveness of the cryotherapy procedure. For instance, the temperature sensed by the temperature sensor can indicate when the target tissue has reached a desired temperature.

In one implementation, the controller 256 can perform a comparison of the temperature sensed by the temperature sensor to a threshold temperature and, based on the comparison, determine that the temperature sensed by the temperature sensor is less than the threshold temperature. The threshold temperature can be a fixed value stored in the memory of the controller 256, or a variable value that is set by a user using the base-station input device 270. Responsive to determining that the temperature sensed by the temperature sensor is approximately equal to the threshold temperature, the controller 256 can cause the output device 272 to provide audio output and/or a visual output to indicate to the medical practitioner that the cryogen 236 that the target tissue has reached the threshold temperature.

In some implementations, the controller 256 can be additionally or alternatively configured to automatically stop a supply and/or reduce a flow rate of the cryogen 236 to the end-effector 262 responsive to the temperature sensor sensing that the temperature is below the threshold temperature. For instance, responsive to the controller 256 determining that the temperature sensed by the temperature sensor is less than the threshold temperature, the controller 256 can automatically cause the cryogen flow assembly 254 of the base station 230 and/or the cryogen flow system 264 of the cryotherapy applicator 232 to stop supplying the cryogen 236 to the end-effector 262 and/or reduce a flow rate of the cryogen 236 to the end-effector 262.

In some implementations in which the cryotherapy applicator 232 includes the temperature sensor on the shaft 260, in the shaft 260, and/or in the end-effector 262, the controller 256 can additionally or alternatively actuate a heater 275 on the shaft 260, in the shaft 260, and/or in the end-effector 262 of the cryotherapy applicator 232. As an example, the heater 275 can include an electric resistive heating device that can transduce electrical energy into thermal energy for heating the shaft 260 and/or the end effector 262. In this arrangement, the controller 256 can be configured to, based on the sensor signal, cause the heater 275 to increase the temperature of the shat 260 and/or the end-effector 262. In one example, the controller 256 can perform a comparison of the temperature sensed by the temperature sensor to a threshold temperature and, based on the comparison, determine that the temperature sensed by the temperature sensor is less than the threshold temperature. Responsive to determining that the temperature sensed by the temperature sensor is less than the threshold temperature, the controller 256 can cause the heater 275 to increase the temperature of the shaft 260 and/or the end-effector 262 until the controller 256 determines that the temperature sensed by the temperature sensor is at or above the threshold temperature.

In some examples, the one or more sensors 274 can be additionally or alternatively configured to sense an amount of the cryogen 236 that is in the canister 242, which is coupled to the canister receptacle 240. For example, the one or more sensors 274 can include a weight sensor that is configured to sense a weight of the canister 242 in the canister receptacle 240 and, based on the weight sensed by the weight sensor, the controller 256 can determine the amount of the cryogen 236 in the canister 242. In another example, the one or more sensors 274 can include a flow rate sensor that can sense a flow rate of the cryogen 236 and, based on the sensed flow rate over a period of time during which the flow rate was sensed, the controller 256 can determine the amount of the cryogen 236 in the canister 242. The output device 272 can be configured to output an indication of the amount of the cryogen 236 in the canister 242 sensed by the one or more sensor(s). This can help the medical practitioner to understand whether the amount of cryogen 236 in the canister 242 is great enough to perform a next cryotherapy procedure.

In one implementation, the controller 256 can perform a comparison of the amount of the cryogen 236 sensed by the one or more sensors 274 with a threshold amount, which is related to an amount of the cryogen 236 that is needed to perform a next cryotherapy procedure. Based on the comparison, the controller 256 can determine that the sensed amount of the cryogen 236 is less than the threshold amount of the cryogen 236. Responsive to determining that the sensed amount of the cryogen 236 is less than the threshold amount, the controller 256 can disable the cryogen flow assembly 254 and/or the cryogen flow system 264 such that a cryotherapy procedure cannot be started until the one or more sensors 274 sense an amount of the cryogen 236 that is greater than the threshold amount (e.g., after another canister 242 is coupled to the canister receptacle 240). This can help to avoid a scenario where a cryotherapy procedure is started without a sufficient amount of the cryogen 236 to complete the cryotherapy procedure.

In some examples, the one or more sensors 274 can be additionally or alternatively configured to sense a pressure of the cryogen 236 in the canister 242, which is coupled to the canister receptacle 240. For example, the one or more sensors 274 can include a pressure sensor that can sense a pressure of the cryogen 236 at or near an interface between the canister 242 and the cryogen flow assembly 254. In some instances, the pressure of the cryogen 236 can affect the flow rate of the cryogen 236. In some implementations, the output device 272 can output an indication of the pressure of the cryogen 236 based on the pressure sensed by the pressure sensor.

In some examples, the one or more sensors 274 can additionally or alternatively include a sensor that can determine information relating to the canister 242 that is coupled to the canister receptacle 240. For example, the canister 242 can include a data storage device (e.g., a non-transitory computer readable medium such as a radiofrequency identification (RFID) tag, and/or an erasable programmable read-only memory (EPROM) chip) and the one or more sensors 274 can include a reader device (e.g., a RFID reader and/or an EPROM reader) that can read information from the data storage device of the canister 242 to determine at least one item of information selected from a group including: (i) a type of the cryogen 236 in the canister 242, (ii) a size of the canister 242, (iii) a shape of the canister 242, (iv) a manufacturer of the canister 242, (v) an amount of the cryogen 236 in the canister 242, and (v) a number of times the cartridge 242 has used.

In another example, the one or more sensors 274 can include a first set of contacts (e.g., mechanical and/or electrical contacts), and the canister 242 can include a second set of contacts (e.g., mechanical and/or electrical contacts) that can engage the first set of contacts when the canister 242 is received in the canister receptacle 240. For instance, the information can be encoded to different arrangements of the second contacts such that different canisters 242 having different information can have different arrangements of the second contacts. Based on the engagement between the contacts, the one or more sensors 274 can determine the information relating to the canister 242.

In some examples, the output device 272 can provide an output to the medical practitioner including the information relating to the canister 242 described above. In some examples, the controller 256 can additionally or alternatively use the information relating to the canister 242 to control operation of the base station 230 (e.g., to perform the comparison of the amount of cryogen 236 to the threshold value as described above).

In some examples, the one or more sensors 274 can additionally or alternatively include an ultrasonic Doppler flow sensor and/or an optical Doppler flow sensor at a distal portion of the shaft 260. The ultrasonic Doppler flow sensor and/or an optical Doppler flow sensor can be used to locate an artery associated with a target tissue. In one example, the artery associated with the target tissue can include at least one nasal nerve and/or an artery from a sphenopalatine branch. In an implementation, the controller 256 can receive the sensor signal from the ultrasonic Doppler flow sensor and/or an optical Doppler flow sensor and, based on the sensor signal, the controller 256 can cause the output device 272 to provide an audio output and/or a visual output that indicates that the end-effector 262 is positioned at the target tissue.

In some examples, the one or more sensors 274 can additionally or alternatively include an electrode sensor at a distal portion of the shaft 260 and/or on the end-effector 262. The electrode sensor can be used to determine that the end-effector 262 is positioned at the target tissue (e.g., at the target nerve), and/or confirm an effectiveness of ablation by determining a change in a physiological response to electrical stimulation, using the electrode sensor, before, during, and/or after ablation. In an implementation, the controller 256 can receive the sensor signal from the electrode sensor and, based on the sensor signal, the controller 256 can cause the output device 272 to provide an audio output and/or a visual output that indicates that the end-effector 262 is positioned at the target tissue (e.g., at the target nerve) and/or confirmation of the effectiveness of ablation.

Also, in an implementation, the electrode sensor can include one or more impedance-based sensors that can measure an impedance of a tissue. For instance, the electrode sensor can include one or more stimulation/response electrodes, and/or a polar electrode array that is configured to measure at least one of: complex impedance and conductivity. In one example, the electrode sensor can be configured to apply an electrical signal (e.g., a 300 mHz signal) and responsively determine, based on a electricla signal, an impedance value. The controller 256 can then receive the sensor signal and determine, based on the impedance value, whether the end-effector 262 is positioned at the target tissue (e.g., at the target nerve), and/or confirm an effectiveness of ablation by determining a change in a physiological response to electrical stimulation.

In some examples, the one or more sensors 274 can additionally or alternatively include a sensor that can determine information relating to the cryotherapy applicator 232 that is coupled to the canister receptacle 240. For example, the cryotherapy applicator 232 and/or the cryogen conduit 234 can include a data storage device (e.g., a non- transitory computer readable medium such as a RFID tag and/or an EPROM chip) and the one or more sensors 274 can include a reader device (e.g., a RFID reader and/or an EPROM reader) that the RFID reader can read information from the data storage device to determine at least one item of information selected from a group including: (i) a size of the cryotherapy applicator 232, (ii) a shape of the cryotherapy applicator 232, and (iii) a manufacturer of the cryotherapy applicator 232. In another example, the one or more sensors 274 and/or the cryotherapy applicator 232 can include the first set of contacts and the second set of contacts, respectively, in a manner similar to that described above with respect to the canister 242. Within examples, the output device 272 can provide an output to the medical practitioner including the information relating to the cryotherapy applicator 232 described above.

As noted above, in some examples, the cryotherapy system 200 can be configured such that the base station 230 can be coupled with a plurality of different cryotherapy applicators 232. In such examples, the cryotherapy system 200 can include the cryotherapy applicator 232 and one or more additional cryotherapy applicators, where at least one of the one or more additional cryotherapy applicators is different from the cryotherapy applicator in at least one of a size or a shape. In such examples, it can be beneficial to indicate, using the output device 272, to the medical practitioner the type of cryotherapy applicator 232 that is coupled to the base station 230. In other examples, the controller 256 can set one or more parameters (e.g., a flow rate of the cryogen 236, a timer for supplying the cryogen 236, an amount of the cryogen 236 to supply, and/or a temperature at which to maintain the canister 242 using the heater 273) for operating the base station 230 based on the information relating to cryotherapy applicator 232 determined using the one or more sensors 274.

In some examples, the controller 256 can use the information relating to the cryotherapy applicator 232 to prevent the cryotherapy applicator 232 from being used during more than one cryotherapy procedure. For instance, the reader device can be further configured to write usage information to the data storage device of the cryotherapy applicator 232 to indicate that the cryotherapy applicator 232 has been used during a first cryotherapy procedure. Prior to performing a second cryotherapy procedure, the reader device can read the usage information from the data storage device and, responsive to a determination that the usage information indicates the cryotherapy applicator 232 was previously used, the controller 256 can prevent the base station 230 from supplying the cryogen 236 for the second cryotherapy procedure.

In some examples, the cryogen 236 that is returned to the base station 230 can be exhausted through a vent to an environment external to the housing 238 of the base station 230. In other examples, the base station 230 can include a cryogen collection reservoir 275 that is configured to receive the cryogen 236 returned from the cryotherapy applicator 232 to the base station 230. The cryogen collection reservoir 275 can be removably coupled to the housing 238 such that the cryogen collection reservoir 275 can be emptied and/or replaced.

In one implementation that includes a cryogen collection reservoir 275, the one or more sensors 274 can be configured to determine when the cryogen collection reservoir 275 should be emptied and/or replaced. For instance, the one or more sensors 274 can sense when an amount of the cryogen 236 in the cryogen collection reservoir is equal to or greater than a threshold amount, and transmit a signal to the controller 256. Based on the signal, the controller 256 can make a determination that the amount of the cryogen 236 in the cryogen collection reservoir 275 is equal to or greater than the threshold amount. In response to the determination, the controller 256 can cause the output device 272 to provide an alarm in the form of an audio output and/or a visual output to indicate to the medical practitioner that the cryogen collection reservoir 275 should be emptied and/or replaced.

As shown in FIG. 2, in some examples, the cryotherapy system 200 can include a camera 276 that can capture an image and generate image data that is representative of the image captured by the camera 276. The camera 276 can be coupled to the cryotherapy applicator 232 such that a field of view of the camera 276 includes anatomy of the patient when the shaft 260 and/or the end-effector 262 are positioned in the body cavity of the patient. In this way, the camera 276 can be used to identify anatomical landmarks, and guide the placement of the end-effector 262 at the target tissue.

In some implementations, the camera 276 can be removably coupled to the cryotherapy applicator 232. For example, the cryotherapy applicator 232 can include a mount 278 that is configured to couple the camera 276 to the cryotherapy applicator 232. As one example, the mount 278 can include a clip that couple the camera 276 to the shaft 260 by a friction-fit coupling. Removably coupling the camera 276 to the cryotherapy applicator 232 can facilitate reuse of the camera 276 in implementations in which other components of the cryotherapy applicator 232 are disposable. In other examples, the camera 276 can be permanently coupled to the cryotherapy applicator 232.

The camera 276 can be in communication with the controller 256. In this example, the output device 272 can include a display device that is communicatively coupled to the controller 256. In this arrangement, the camera 276 can communicate the image data to the controller 256, and the controller 256 can use the image data to cause the display device to display the image captured by the camera 276. This arrangement can provide a convenient way for the medical practitioner to view images of the patient's anatomy while navigating the end-effector 262 to the target tissue.

Additionally, in some examples, the base station 230 can include one or more components of the camera 276, which can help to reduce a size of a portion of the camera 276 that is inserted into the body cavity with the cryotherapy applicator 232. For instance, a power source of the camera 276 can be located in the base station 230.

In some examples, the cryotherapy system 200 can additionally or alternatively include one or more features that can enhance a lighting condition in the body cavity to help visualize the patient's anatomy with the camera 276. For instance, as shown in FIG. 2, the base station 230 can include a light source 280 that is configured to generate light. The cryotherapy system 200 can also include an optical connector 282 that is configured to transmit the light to the camera 276 to illuminate the field of view of the camera 276. As an example, the optical connector 282 can be a fiber optic cable.

As shown in FIG. 2, one or more optical elements 284 can be coupled to the optical connector 282 at the camera 276. The one or more optical elements 284 can include one or more structures that facilitate emitting the light from the optical connector 282 to illuminate the field of view of the camera 276. As examples, the one or more optical elements 284 can include at least one optical element selected from a group including: a lens, a grating, a prism, a faceted surface, an optical filter, and/or a reflective surface. In one implementation, the one or more optical elements 284 can be positioned around a circumference of the camera 276 such that the optical elements 284 can emit the light around the circumference of the camera 276. This can help to reduce or eliminate shadows within the field of view of the camera 276.

As noted above, the cryogen conduit 234 can supply the cryogen 236 from the base station 230 to the cryotherapy applicator 232. In some examples, the cryogen conduit 234 can also return the cryogen 236 from the end-effector 262 to the base station 230. For instance, in one example, the cryogen conduit 234 can include a first lumen for supplying the cryogen 236 from the base station 230 to the cryotherapy applicator 232, and a second lumen for returning the cryogen 236 from the cryotherapy applicator 232 to the base station 230. In one implementation, the first lumen and the second lumen can be coaxial with each other (e.g., the first lumen can be positioned in the second lumen, or the second lumen can be positioned in the first lumen). In another example, the first lumen and the second lumen can be in a side-by-side arrangement.

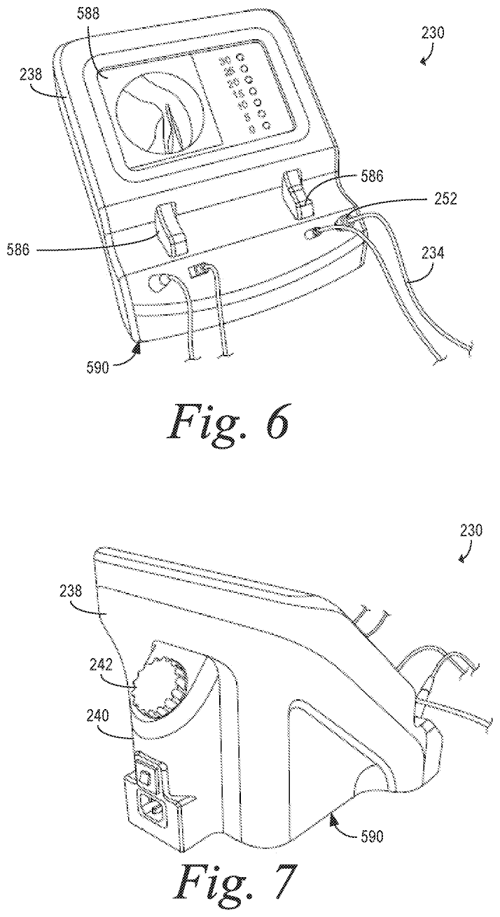

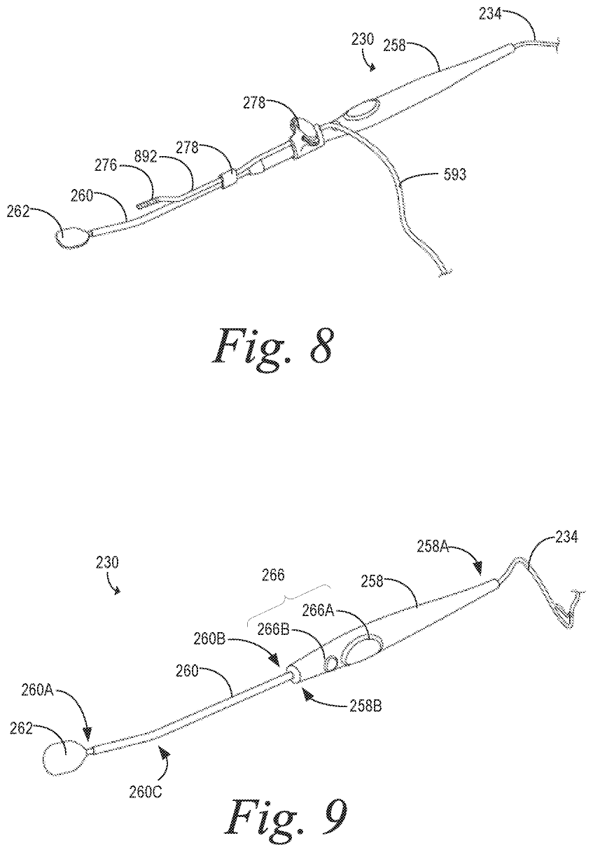

FIGS. 5-9 depict one example implementation of the cryotherapy system 200 shown in FIG. 2 and described above. In particular, FIG. 5 depicts a perspective view of the base station 230, the cryotherapy applicator 232, and the camera 276, FIG. 6 depicts a front side of the base station 230, FIG. 7 depicts a back side of the base station 230, FIG. 8 depicts a perspective view of the camera 276 coupled to the cryotherapy applicator 232, and FIG. 9 depicts a perspective view of the cryotherapy applicator 232 without the camera 276, according to the example implementation.

As shown in FIGS. 5-7, the base station 230 includes the housing 238. The housing 238 can define an internal chamber in which one or more components of the cryotherapy system 200 can be housed, as described above. In the example implementation shown in FIGS. 5-6, the housing 238 includes a cradle 586 that is configured to receive the cryotherapy applicator 232 when the cryotherapy applicator 232 is not in use. This can help to protect the cryotherapy applicator 232 and/or the camera 276 when the cryotherapy applicator 232 and/or the camera 276 are not in use. Additionally, as shown in FIGS. 5-6, the base station 230 includes a display device 588 that provides at least one of the functions described above with respect to the base-station input device 270 and/or the output device 272.

In FIGS. 5-7, a bottom surface 590 of the housing 238 can be configured to rest in a stabile position on a substantially flat support surface (e.g., of a table, a counter, a desk, a shelf, and/or a medical equipment cart). For instance, the bottom surface 590 of the housing 238 can be a substantially planar surface and/or the bottom surface 590 can include one or more adjustable support elements that can help to balance the base station 230 on the support surface. This can be beneficial as the cryotherapy applicator 232 is moved relative to the base station 230 during a cryotherapy procedure.

As shown in FIG. 7, the base station 230 includes the canister receptacle 240 on the back side of the housing 238. However, the canister receptacle 240 can be in a different location in other examples. Also, in FIG. 7, a canister 242 is coupled to the canister receptacle 240. In this example, when the canister 242 is coupled to the canister receptacle 240, a outlet of the canister 242 (e.g., through which the cryogen 236 exits the canister 242) is oriented in an upright position relative to ground (e.g., substantially parallel to a direction of gravity). This can help to mitigate a gas in the canister 236 entering the cryogen flow assembly 254.

As shown in FIGS. 5-9, the cryotherapy applicator 232 can be coupled to the cryogen outlet 252 by the cryogen conduit 234. The cryogen conduit 234 has (i) a first end extending from the proximal end of the handle 258 of the cryotherapy applicator 232 and (ii) a second end that is coupled to the cryogen outlet 252 of the base station 230. In this arrangement, the cryogen conduit 234 can supply the cryogen 236 from the base station 230 to the cryotherapy applicator 232 and/or return the cryogen 236 from the cryotherapy applicator 232 to the base station 230 as described above.

As shown in FIGS. 8 and 9, the cryotherapy applicator 232 includes the handle 258, the shaft 260, and the end-effector 262. The handle 258 can be gripped by a user during a cryotherapy procedure. As shown in FIG. 9, the handle 258 has a proximal end 258A and a distal end 258B. The shaft 260 extends from the distal end 258B of the handle 258. The end-effector 262 coupled to a distal end 260A the shaft 260. In FIGS. 5, 8, and 9, the end-effector 262 includes a balloon that is configured to be inflated when the cryogen 236 is supplied to the end-effector 262. However, the end-effector 262 can have a different configuration in other examples.

As shown in FIG. 9, between the distal end 260A of the shaft 260 and a proximal end 260B of the shaft 260, the shaft 260 can include a bend at a curved portion 260C. In some implementations, the curved portion 260C of the shaft 260 can help to navigate the end-effector 262 through the body cavity (e.g., through a cavity in the nose, the ear, and/or the throat of the patient) and around anatomical structures in the body cavity.

As noted above, the base station 230 can be configured to couple to a plurality of cryotherapy applicators 232, where each cryotherapy applicator 232 is different from another cryotherapy applicator 232 with respect to at least one of a size or a shape of the cryotherapy applicator 232. In one example, the plurality of cryotherapy applicators 232 can each have a respective curved portion 260C that differs from another of the cryotherapy applicators 232 in an angle formed by the curved portion 260C between a distal portion of the shaft 260 (e.g., a portion between the distal end 260A and the curved portion 260C) and a proximal portion of the shaft 260 (e.g., a portion between the proximal end 260B and the curved portion 260C). In another example, the plurality of cryotherapy applicators 232 can additionally or alternatively each have a respective length of the shaft 260 between the proximal end 260B and the distal end 260A, where the respective lengths differ from each other. In another example, a size and/or a shape of the end-effector 262 of at least one of the cryotherapy applicators 232 can be different than a size and/or a shape of the end-effector 262 of a least another one of the cryotherapy applicators 232.

In some examples, the plurality of cryotherapy applicators 232 can be packaged and sold as a kit, where each cryotherapy applicator 232 in the kit differs from at least another cryotherapy applicator 232 in the kit with respect to at least one of: a size of the handle 258, a shape of the handle 258, a size of the shaft 260, a shape of the shaft 260, a size of the end-effector 262, a shape of the end-effector 262, and a type of end-effector 262 (e.g., a balloon-type end-effector 262, a plate-type end-effector 262, an omni-directional end-effector 262, and/or an end-effector 262 with an active surface and an inactive surface).

As shown in FIG. 9, the cryotherapy applicator 232 includes the user input device 266 on the handle 258. In FIG. 9, the user input device 266 includes a first button 262A and a second button 262B. In this example, the first button 262A is operable to start and stop the flow of the cryogen 236 to the end-effector 262. The second button 262B can be operable to activate the heater 273 in the base station 230, the light source 280 in the base station 230, and/or the camera 276. Although the user input device 266 includes the first button 262A and the second button 262B in FIG. 9, the user input device 266 can include a fewer quantity or a greater quantity of buttons in other examples. Additionally, as described above, the user input device 266 can include other types of devices in addition or alterative to buttons in other examples.

FIG. 8 shows the camera 276 coupled to the end-effector 262, according to an example. The camera 276 can be at a distal end of a camera shaft 892. At least a portion of the camera shaft 892 can be approximately parallel to the shaft 260 of the cryotherapy applicator 232. In some examples, the camera 276 can be at a fixed position relative to the cryotherapy applicator 232 when the camera 276 is coupled to the cryotherapy applicator 232.

In other examples, a distal portion of the camera shaft 892 can be movable relative to a proximal portion of the camera shaft 892 to facilitate adjusting the field of view of the camera 276 relative to the cryotherapy applicator 232. For instance, the camera shaft 892 can be malleable such that the user can manually adjust a position and/or an orientation of the camera 276 relative to the cryotherapy applicator 232 prior to inserting the cryotherapy applicator 232 and the camera 276 in the body cavity during the cryotherapy procedure. In another implementation, the distal portion of the camera shaft 892 can be movable relative to the proximal portion of the camera shaft 892 while the cryotherapy applicator 232 and the camera 276 are positioned in the body cavity. For instance, the camera 276 can include one or more pull wires that can be operated to move the distal portion of the camera shaft 892 relative to the proximal portion of the camera shaft 892 and, thus, adjust the field of view of the camera 276.

As shown in FIG. 8, the camera 276 can be coupled to the end-effector 262 by the mount 278. In this example, the mount 278 includes a clip that couples the camera shaft 892 to the shaft 260 of the cryotherapy applicator 232. However, in other examples, the mount 278 can have a different configuration and/or the camera can be integrated into the handle 258 and/or the shaft 260 of the cryotherapy applicator 232. By coupling camera 276 to the cryotherapy applicator 232, the user can conveniently hold the handle 258 of the cryotherapy applicator 232 to support and maneuver both the cryotherapy applicator 232 and the camera 276. This can advantageously allow the user to operate both the cryotherapy applicator 232 and the camera 276 with a single hand (e.g., in contrast to existing cryotherapy systems).

As shown in FIGS. 5 and 8, the camera 276 can include a data connector 593 configured to transmit the image data that is representative of the image captured by the camera 276. As shown in FIG. 5, the base station 230 can include an optical input 594 that can couple the data connector 593 of the camera 276 to the controller 256 of the base station 230. In this arrangement, the controller 256 can use the image data, which is received from the camera 276 via the data connector 593, to cause the display device 588 to display the image captured by the camera 276.

Although the camera 276 is communicatively coupled to the controller 256 by the data connector 593 in FIGS. 5-9, the camera 276 can be in wireless communication with the controller 256 in other examples.

FIG. 5 also shows the optical connector 282 coupled to the base station 230 at a light output port 596. Additionally, in FIG. 5, the base station 230 includes a data port 597 that can couple the base station 230 to an external computing device (not shown). For example, the data port 597 can be an Ethernet port and/or a universal serial bus (USB) port that can provide for wired communication between one or more of the components of the base station 230 with the external computing device. As an example, the external computing device can be a computing system of a healthcare provider and/or hospital. In some examples, the base station 230 can be additionally or alternatively in wireless communication with the external computing device.

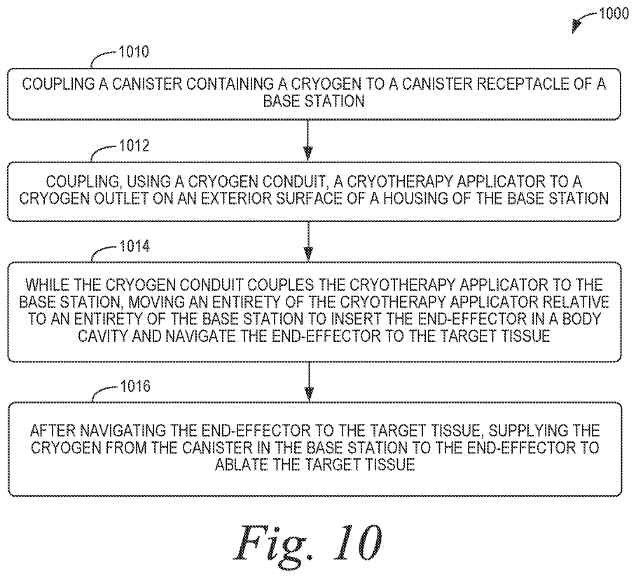

Referring now to FIG. 10, a flowchart for a process 1000 of operating a cryotherapy system is depicted according to an example. At block 1010, the process 800 can include coupling a canister containing a cryogen to a canister receptacle of a base station. At block 1012, the process 1000 can include coupling, using a cryogen conduit, a cryotherapy applicator to a cryogen outlet on an exterior surface of a housing of the base station. The cryogen conduit has (i) a first end extending from a proximal end of a handle of the cryotherapy applicator and (ii) a second end configured to couple to the cryogen outlet of the base station.

The cryotherapy applicator includes (i) the handle that is configured to be gripped by a user during a cryotherapy procedure, wherein the handle has the proximal end and a distal end, (ii) a shaft extending from the distal end of the handle, and (iii) an end-effector coupled to the shaft, wherein the end-effector is configured to use the cryogen to ablate a target tissue.

At block 1014, the process 1000 includes, while the cryogen conduit couples the cryotherapy applicator to the base station, moving an entirety of the cryotherapy applicator relative to an entirety of the base station to insert the end-effector in a body cavity and navigate the end-effector to the target tissue. The body cavity includes a cavity in an ear, a nose, or a throat. At block 1016, the process 1000 includes, after navigating the end-effector to the target tissue, supplying the cryogen from the canister in the base station to the end-effector to ablate the target tissue.

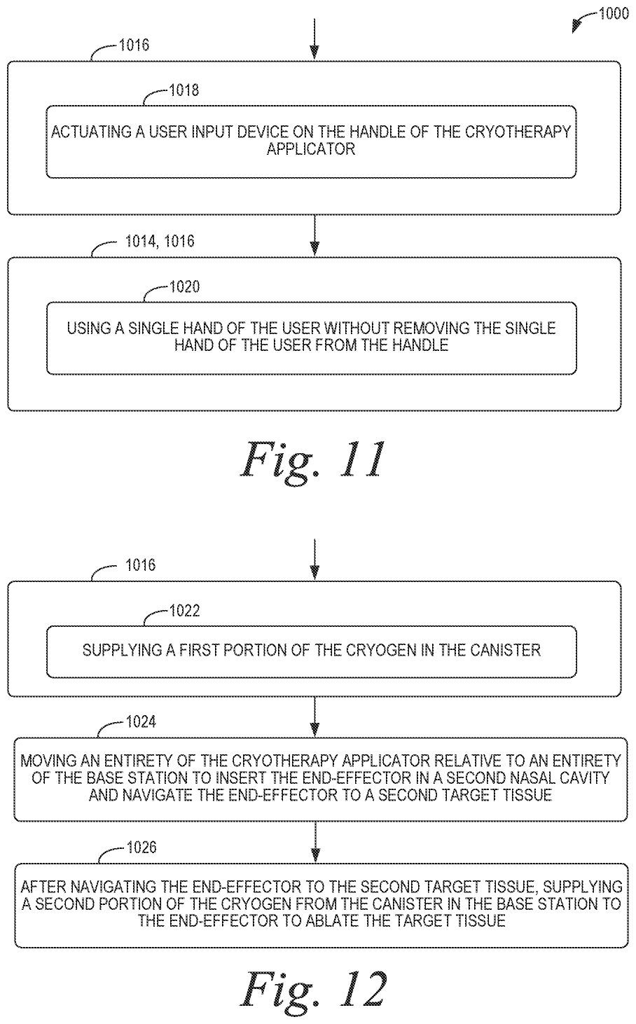

FIGS. 11-16 depict additional aspects of the process 1000 according to further examples. As shown in FIG. 11, supplying the cryogen at block 1016 is responsive to actuating a user input device on the handle of the cryotherapy applicator at block 1018. Also, in FIG. 11, moving the entirety of the cryotherapy applicator relative to the entirety of the base station at block 1014 and actuating the user input device on the handle of the cryotherapy applicator at block 1016 are both performed using a single hand of the user without removing the single hand of the user from the handle at block 1020.

As shown in FIG. 12, supplying the cryogen from the canister in the base station to the end-effector to ablate the target tissue at block 1016 can include supplying a first portion of the cryogen in the canister at block 1022. In FIG. 12, the process 1000 can also include, after ablating the target tissue using the first portion of the cryogen in the canister: (i) moving an entirety of the cryotherapy applicator relative to an entirety of the base station to insert the end-effector in a second nasal cavity and navigate the end-effector to a second target tissue at block 1024, and (ii) after navigating the end-effector to the second target tissue at block 1024, supplying a second portion of the cryogen from the canister in the base station to the end-effector to ablate the target tissue at block 1026.

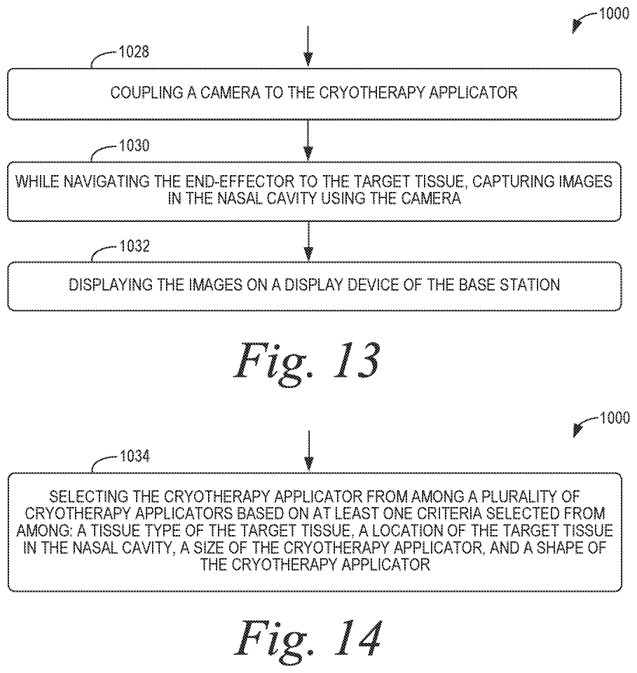

As shown in FIG. 13, the process 1000 can also include coupling a camera to the cryotherapy applicator at block 1028. In FIG. 13, the process 1000 can further include, while navigating the end-effector to the target tissue at block 1014, capturing images in the nasal cavity using the camera at block 1030. Additionally, the process 1000 can include displaying the images on a display device of the base station at block 1032.

As shown in FIG. 14, the process 1000 can also include, at block 1034, selecting the cryotherapy applicator from among a plurality of cryotherapy applicators based on at least one criteria selected from among: a tissue type of the target tissue, a location of the target tissue in the nasal cavity, a size of the cryotherapy applicator, and a shape of the cryotherapy applicator.

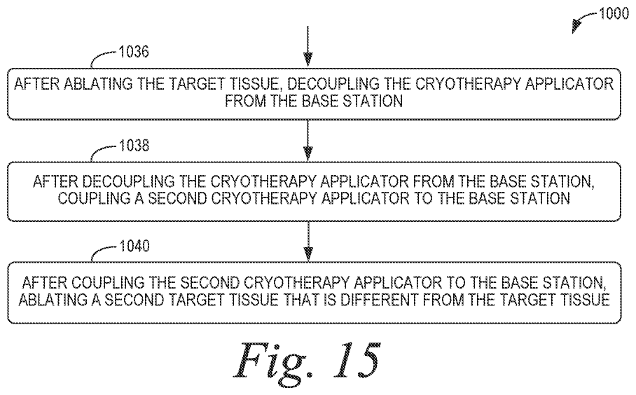

As shown in FIG. 15, the process 1000 can also include, after ablating the target tissue at block 1016, decoupling the cryotherapy applicator from the base station at block 1036. The process 1000 can further include, after decoupling the cryotherapy applicator from the base station at block 1036, coupling a second cryotherapy applicator to the base station at block 1038. After coupling the second cryotherapy applicator to the base station at block 1038, the process 1000 can include ablating a second target tissue that is different from the target tissue at block 1040.

The description of the different advantageous arrangements has been presented for purposes of illustration and description, and is not intended to be exhaustive or limited to the embodiments in the form disclosed. Many modifications and variations will be apparent to those of ordinary skill in the art. Further, different advantageous embodiments may describe different advantages as compared to other advantageous embodiments. The embodiment or embodiments selected are chosen and described in order to explain the principles of the embodiments, the practical application, and to enable others of ordinary skill in the art to understand the disclosure for various embodiments with various modifications as are suited to the particular use contemplated.

Any of the blocks shown in FIGS. 10-15 may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In some instances, components of the devices and/or systems described herein may be configured to perform the functions such that the components are actually configured and structured (with hardware and/or software) to enable such performance. Example configurations then include one or more processors executing instructions to cause the system to perform the functions. Similarly, components of the devices and/or systems may be configured so as to be arranged or adapted to, capable of, or suited for performing the functions, such as when operated in a specific manner.

Although FIGS. 2-9 depict a cryotherapy system 200, the concepts of the present application can be applied to other modalities for ablation of a target tissue. Indeed, the concepts described herein can be extended to an ablation system that can ablate a target tissue using radiofrequency (RF) energy, ultrasonic energy, and/or heat. For instance, an ablation system can include a radiofrequency energy generator and a radiofrequency applicator in addition or alternative to any or all of the components of the cryotherapy system 200 described above. In one implementation, the canister 242, the canister receptacle 240, the cryogen flow assembly 254, the cryogen conduit 234, and the cryogen flow system 264 can be replaced with electrical circuit components (e.g., one or more conductors, switches, transistors, operational amplifiers, etc.) that are configured to supply the radiofrequency energy to the end-effector 262, which uses the RF energy to ablate the target tissue. In this implementation, the other components of the system can function in a manner similar to that described above with respect to the other components of the cryotherapy system 200 described above. The cryotherapy system 200 can be adapted in a similar manner to ablate tissue via ultrasonic energy and/or heat.

Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

The breadth of the present application is not to be limited by the subject specification, but rather only by the plain meaning of the claim terms employed.

What is claimed is:

1. A cryotherapy system, comprising:
a base station comprising:
a housing including a canister receptacle that is configured to receive a canister containing a cryogen, wherein the housing defines an internal chamber,
a cryogen outlet on an exterior surface of the housing, wherein the cryogen outlet is configured to output the cryogen from the base station,
a cryogen flow assembly in the internal chamber of the housing, wherein the cryogen flow assembly is configured to supply the cryogen from the canister to the cryogen outlet, and
a controller configured to control a flow of the cryogen through the cryogen flow assembly from the canister to the cryogen outlet;
a cryotherapy applicator comprising:
a handle that is configured to be gripped by a user during a cryotherapy procedure, wherein the handle has a proximal end and a distal end,
a shaft extending from the distal end of the handle, and
an end-effector coupled to the shaft, wherein the end-effector is configured to use the cryogen to ablate a target tissue; and
a cryogen conduit configured to couple the cryotherapy applicator to the base station and supply the cryogen from the base station to the cryotherapy applicator, wherein the cryogen conduit has (i) a first end extending from the proximal end of the handle of the cryotherapy applicator and (ii) a second end configured to couple to the cryogen outlet of the base station,
wherein an entirety of the cryotherapy applicator is movable relative to an entirety of the base station while the cryogen conduit couples the cryotherapy applicator to the base station.

2. The cryotherapy system of claim 1, wherein the canister receptacle is further configured to receive a second canister, which has a size that is greater than a size of the canister,
wherein the canister contains a first volume of the cryogen and the second canister contains a second volume of the cryogen, and
wherein the second volume is greater than the first volume.

3. The cryotherapy system of claim 1, wherein the canister is configured to contain between approximately 10 milliliters and approximately 32 milliliters of the cryogen.

4. The cryotherapy system of claim 1, further comprising one or more additional cryotherapy applicators, wherein each of the one or more additional cryotherapy applicators are configured to be coupled to the cryogen outlet of the base station.

5. The cryotherapy system of claim 4, wherein at least one of the one or more additional cryotherapy applicators is different from the cryotherapy applicator in at least one of a size of the cryotherapy applicator or a shape of the cryotherapy applicator.

6. The cryotherapy system of claim 1, wherein the base station further comprises one or more sensors configured to sense at least one parameter selected from among: a pressure in the canister and a flow rate of the cryogen in the cryogen flow assembly.

7. The cryotherapy system of claim 6, wherein the base station comprises a heater that is configured to increase a temperature of the canister in the canister receptacle,
wherein the one or more sensors are configured to transmit to the controller a sensor signal indicative of the at least one parameter sensed by the one or more sensors, and
wherein the controller is configured to, based on the sensor signal, cause the heater to increase the temperature of the canister of the canister receptacle.

8. The cryotherapy system of claim 1, wherein the cryotherapy applicator comprises a mount configured to couple a camera to the cryotherapy applicator,
wherein the camera comprises a connector configured to transmit image data that is representative of an image captured by the camera,
wherein the base station comprises an optical input configured to couple the connector of the camera to the controller of the base station, and
wherein the base station comprises a display device that is communicatively coupled to the controller, and
wherein the controller is configured to use the image data to cause the display device to display the image captured by the camera.

9. The cryotherapy system of claim 8, wherein the base station comprises a light source that is configured to generate light, and
wherein the connector is configured to transmit the light to the camera to illuminate a field of view of the camera.

10. The cryotherapy system of claim 1, wherein the cryotherapy applicator further comprises a user input device on the handle, wherein the user input device is configured to transmit to a control signal to the controller to cause the controller to control the flow of the cryogen.

11. The cryotherapy system of claim 1, further comprising a foot pedal in communication with the controller of the base station, wherein the foot pedal is configured to transmit a control signal to the controller cause the controller to control the flow of the cryogen.

12. The cryotherapy system of claim 1, wherein the handle of the cryotherapy applicator is elongated along a longitudinal axis such that the handle is configured to be held by the user using a pencil grip.

13. The cryotherapy system of claim 1, wherein the base station comprises a base-station input device that is configured to receive one or more inputs from the user, and
wherein the controller is configured to perform one or more actions responsive to the one or more inputs received via the base-station input device.

14. A method operating a cryotherapy system, comprising:
coupling a canister containing a cryogen to a canister receptacle of a base station;
coupling, using a cryogen conduit, a cryotherapy applicator to a cryogen outlet on an exterior surface of a housing of the base station, wherein the cryogen conduit has (i) a first end extending from a proximal end of a handle of the cryotherapy applicator and (ii) a second end configured to couple to the cryogen outlet of the base station, wherein the cryotherapy applicator comprises:
the handle that is configured to be gripped by a user during a cryotherapy procedure, wherein the handle has the proximal end and a distal end,
a shaft extending from the distal end of the handle, and
an end-effector coupled to the shaft, wherein the end-effector is configured to use the cryogen to ablate a target tissue;
while the cryogen conduit couples the cryotherapy applicator to the base station, moving an entirety of the cryotherapy applicator relative to an entirety of the base station to insert the end-effector in a nasal cavity and navigate the end-effector to the target tissue; and
after navigating the end-effector to the target tissue, supplying the cryogen from the canister in the base station to the end-effector to ablate the target tissue.

15. The method of claim 14, wherein supplying the cryogen is responsive to actuating a user input device on the handle of the cryotherapy applicator, and
wherein (i) moving the entirety of the cryotherapy applicator relative to the entirety of the base station and (ii) actuating the user input device on the handle of the cryotherapy applicator are both performed using a single hand of the user without removing the single hand of the user from the handle.

16. The method of claim 14, wherein supplying the cryogen from the canister in the base station to the end-effector to ablate the target tissue comprises supplying a first portion of the cryogen in the canister, and wherein the method further comprises:
after ablating the target tissue using the first portion of the cryogen in the canister:
moving an entirety of the cryotherapy applicator relative to an entirety of the base station to insert the end-effector in a second nasal cavity and navigate the end-effector to a second target tissue; and
after navigating the end-effector to the second target tissue, supplying a second portion of the cryogen from the canister in the base station to the end-effector to ablate the target tissue.

17. The method of claim 14, further comprising:
coupling a camera to the cryotherapy applicator;
while navigating the end-effector to the target tissue, capturing images in the nasal cavity using the camera; and
displaying the images on a display device of the base station.

18. The method of claim 14, further comprising selecting the cryotherapy applicator from among a plurality of cryotherapy applicators based on at least one criteria selected from among: a tissue type of the target tissue, a location of the target tissue in the nasal cavity, a size of the cryotherapy applicator, and a shape of the cryotherapy applicator.

19. The method of claim 14, further comprising:
after ablating the target tissue, decoupling the cryotherapy applicator from the base station;
after decoupling the cryotherapy applicator from the base station, coupling a second cryotherapy applicator to the base station; and
after coupling the second cryotherapy applicator to the base station, ablating a second target tissue that is different from the target tissue.

20. The method of claim 19, wherein the second cryotherapy applicator is different from the cryotherapy applicator in at least one of: a size and a shape.
